(19) United States
(12) Patent Application Publication
(10) Pub. No.: US 2024/0266019 A1
(43) Pub. Date: Aug. 8, 2024

(54) CELL THERAPY SUPPORT APPARATUS, CELL THERAPY SUPPORT SYSTEM, AND CELL THERAPY SUPPORT METHOD

(71) Applicants: Canon Medical Systems Corporation, Otawara-shi (JP); CANON KABUSHIKI KAISHA, Tokyo (JP)

(72) Inventors: Koji HIRATA, Kasukabe (JP); Kohei WATANABE, Utsunomiya (JP)

(73) Assignees: Canon Medical Systems Corporation, Otawara-shi (JP); CANON KABUSHIKI KAISHA, Tokyo (JP)

(21) Appl. No.: 18/431,107

(22) Filed: Feb. 2, 2024

(30) Foreign Application Priority Data
Feb. 3, 2023 (JP) 2023-015598

(51) Int. Cl. G16H 20/17

(52) U.S. Cl. CPC G16H 20/17

(57) ABSTRACT

According to one embodiment, a cell therapy support apparatus includes processing circuitry. The processing circuitry acquires, for each of a first condition and a second condition differing from each other in a degree of inflammation of a target subject, feature information indicating a degree of activation of lymphoid lineage cells in a tissue containing stem cells of the target subject.

The processing circuitry determines a quantity of the tissue to be taken from the target subject based on a difference between the feature information in the first condition and the feature information in the second condition.

### CROSS-REFERENCE TO RELATED APPLICATION

This application is based upon and claims the benefit of priority from Japanese Patent Application No. 2023-015598, filed Feb. 3, 2023, the entire contents of which are incorporated herein by reference.

### FIELD

Embodiments described herein relate generally to a cell therapy support apparatus, a cell therapy support system, and a cell therapy support method.

### BACKGROUND

Cell therapy is a treatment method for treating a patient's disease using cells (e.g., stem cells, immune cells, blood cells) harvested from the patient or a person other than the patient. Normally, harvested cells include both “high-quality” cells suitable for implantation and “low-quality” cells unsuitable for implantation at a certain ratio. Thus, harvesting as many “high-quality” cells as possible from a patient is required for better performance of cell implantation.

In particular, if the same quantities of stem cells are harvested from patients, the performance of the implantation of the stem cells harvested from a patient in an inflammatory condition tends to be lower (e.g., low proliferation rate, low survival rate, low engraftment rate) than the performance of the implantation of the stem cells harvested from a patient in a healthy condition. Therefore, it is desired that the quality of the stem cells of a patient in a current condition be estimated based on a predetermined indicator and then suitable quantities of stem cells be harvested from the patient.

### BRIEF DESCRIPTION OF THE DRAWINGS

FIG. 1 is a block diagram showing an example of a configuration of a cell therapy support system according to an embodiment.

FIG. 2 is a block diagram showing an example of a configuration of a cell therapy support apparatus according to an embodiment.

FIG. 3 is a block diagram showing an example of a configuration of an operation terminal according to an embodiment.

FIG. 4 is a flowchart showing an example of an entire operation of the cell therapy support apparatus according to the embodiment.

FIG. 5 is a flowchart showing an example of a detailed operation of the cell therapy support apparatus according to the embodiment.

FIG. 6 is a diagram showing examples of cytograms obtained in a healthy condition and in an inflammatory condition according to an embodiment.

FIG. 7 is a diagram showing a first example of processing performed by the cell therapy support apparatus according to the embodiment.

FIG. 8 is a diagram showing a second example of processing performed by the cell therapy support apparatus according to the embodiment.

### DETAILED DESCRIPTION

In general, according to one embodiment, a cell therapy support apparatus includes processing circuitry. The processing circuitry acquires, for each of a first condition and a second condition differing from each other in a degree of inflammation of a target subject, feature information indicating a degree of activation of lymphoid lineage cells in a tissue containing stem cells of the target subject. The processing circuitry determines a quantity of the tissue to be taken from the target subject based on a difference between the feature information in the first condition and the feature information in the second condition.

Hereinafter, a cell therapy support apparatus, a cell therapy support system, and a cell therapy support method according to the embodiments will be described with reference to the accompanying drawings. In the embodiments described below, elements assigned with the same reference symbols perform the same operations, and repeat descriptions will be omitted as appropriate.

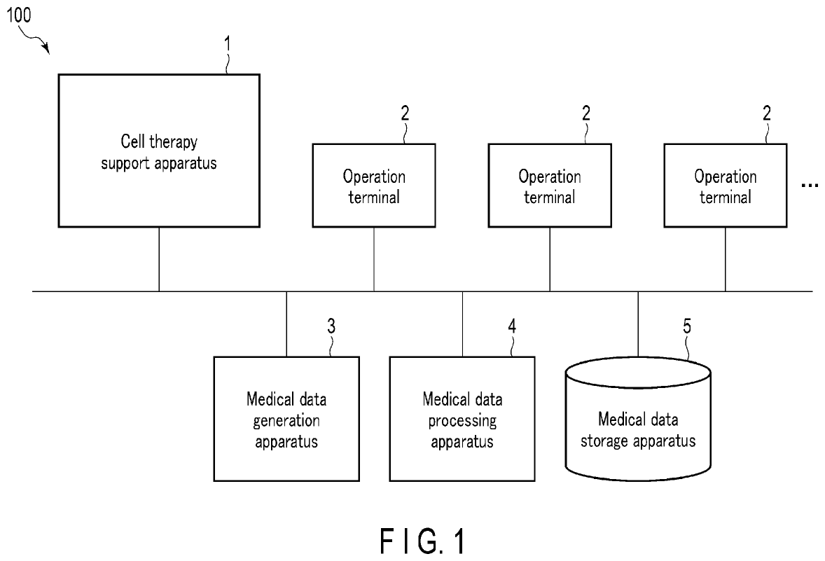

FIG. 1 is a block diagram showing an example of a configuration of a cell therapy support system 100 according to an embodiment. The cell therapy support system 100 is a system for supporting cell therapy. For example, the cell therapy support system 100 is a client server system. The cell therapy support system 100 includes, as its components, a cell therapy support apparatus 1, multiple operation terminals 2, a medical data generation apparatus 3, a medical data processing apparatus 4, and a medical data storage apparatus 5. These components are connected to one another via a bus, which is a common signal transmission path, in such a manner as to be able to communicate with one another.

The cell therapy support apparatus 1 is an apparatus for supporting cell therapy. The cell therapy support apparatus 1 functions as a server in the cell therapy support system 100. The cell therapy support apparatus 1 may be a workstation capable of performing high-speed information processing. The cell therapy support apparatus 1 is also referred to as a “medical information processing apparatus”.

The operation terminal 2 is a terminal operated by an operator (e.g., a nurse). The operation terminal 2 functions as a client in the cell therapy support system 100. The operation terminal 2 may be a desktop PC, a laptop PC, a smartphone, a tablet terminal, or a wearable terminal.

The medical data generation apparatus 3 is an apparatus that generates medical data (e.g., medical image data, vital sign data). The medical data generation apparatus 3 may be a medical diagnostic imaging apparatus (e.g., an X-ray diagnostic apparatus, an X-ray CT apparatus, an MRI apparatus, an ultrasonic diagnostic apparatus, a nuclear medicine examination apparatus) or a wearable device (e.g., a blood-pressure meter or a heart rate meter). The medical data generation apparatus 3 transmits the generated medical data to the medical data processing apparatus 4.

The medical data processing apparatus 4 is an apparatus that processes medical data. For example, the medical data processing apparatus 4 reconstructs, analyzes, or processes medical image data or vital sign data. The medical data processing apparatus 4 functions as a server in the cell therapy support system 100. The medical data processing apparatus 4 may be a workstation capable of performing high-speed information processing. The medical data processing apparatus 4 transmits the processed medical data to the medical data storage apparatus 5.

The medical data storage apparatus 5 is an apparatus that stores medical data. The medical data storage apparatus 5 may be a storage medium (e.g., a magnetic storage medium, an electromagnetic storage medium, an optical storage medium, a semiconductor memory) or a drive that reads and writes information to and from a storage medium. The medical data storage apparatus 5 stores the medical data transmitted from the medical data processing apparatus 4.

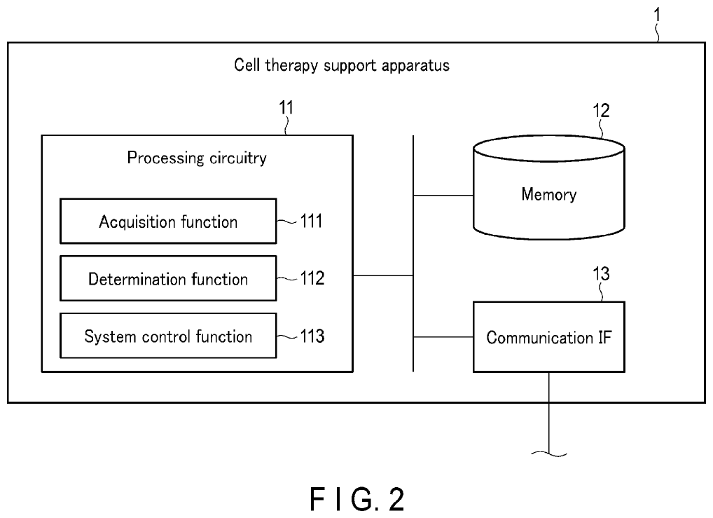

FIG. 2 is a block diagram showing an example of a configuration of the cell therapy support apparatus 1 according to the embodiment. The cell therapy support apparatus 1 includes processing circuitry 11, a memory 12, and a communication IF 13 as its components. These components are connected to one another via a bus, which is a common signal transmission path, in such a manner as to be able to communicate with one another.

The processing circuitry 11 is circuitry that controls the entire operation of the cell therapy support apparatus 1. The processing circuitry 11 includes at least one processor. The processor refers to circuitry such as a CPU (central processing unit), a GPU (graphics processing unit), an ASIC (application specific integrated circuit), or a programmable logic device (e.g., an SPLD (simple programmable logic device), a CPLD (complex programmable logic device), or an FPGA (field programmable gate array)). If the processor is a CPU, the CPU reads and executes the programs stored in the memory 12 to implement the functions. If the processor is an ASIC, each function is directly incorporated as logic circuitry into the circuitry of the ASIC. The processor may be constituted in the form of single circuitry or in the form of multiple independent sets of circuitry combined. The processing circuitry 11 implements each function (an acquisition function 111, a determination function 112, and a system control function 113).

The acquisition function 111 is a function of acquiring various kinds of data or information. Firstly, the acquisition function 111 acquires, for each of a first condition and a second condition differing from each other in the degree of inflammation of a target subject (e.g., a patient, a person other than the patient), feature information C indicating the degree of activation of lymphoid lineage cells (e.g., T cells, B cells, NK cells) in a tissue containing stem cells of the target subject. Secondly, the acquisition function 111 acquires a standard quantity or an upper limit quantity of the tissue to be taken from the target subject. The acquisition function 111 may acquire various kinds of data or information from the medical data storage apparatus 5 by accessing the medical data storage apparatus 5. The acquisition function 111 is an example of an acquisition unit.

The determination function 112 is a function of determining various contents. Firstly, the determination function 112 determines a quantity of a tissue to be taken from the target subject based on a difference between the feature information C in the first condition and the feature information C in the second condition. Secondly, the determination function 112 corrects the standard quantity of tissue sampling based on the same difference and thereby determines a quantity of tissue sampling. Thirdly, the determination function 112 causes a warning to be displayed on a display if the quantity of tissue sampling exceeds the upper limit quantity. The determination function 112 is an example of a determination unit.

The system control function 113 is a function of controlling various operations performed by the processing circuitry 11. For example, the system control function 113 provides an operating system (OS) for the processing circuitry 11 to implement each function (the acquisition function 111 and the determination function 112). The system control function 113 is an example of a system controller.

The memory 12 is a device for storing various kinds of data or information. The memory 12 may be a processor-readable storage medium (e.g., a magnetic storage medium, an electromagnetic storage medium, an optical storage medium, a semiconductor memory) or a drive that reads and writes data or information to and from a storage medium. The memory 12 stores each program that causes the processing circuitry 11 to implement each function (the acquisition function 111, the determination function 112, and the system control function 113). The memory 12 is an example of a storage unit.

The communication IF 13 is an interface that communicates various kinds of data or information with each of the components included in the cell therapy support system 100. The communication IF 13 communicates various kinds of data or information with the operation terminals 2 or the medical data storage apparatus 5. The communication IF 13 is an example of a communication unit.

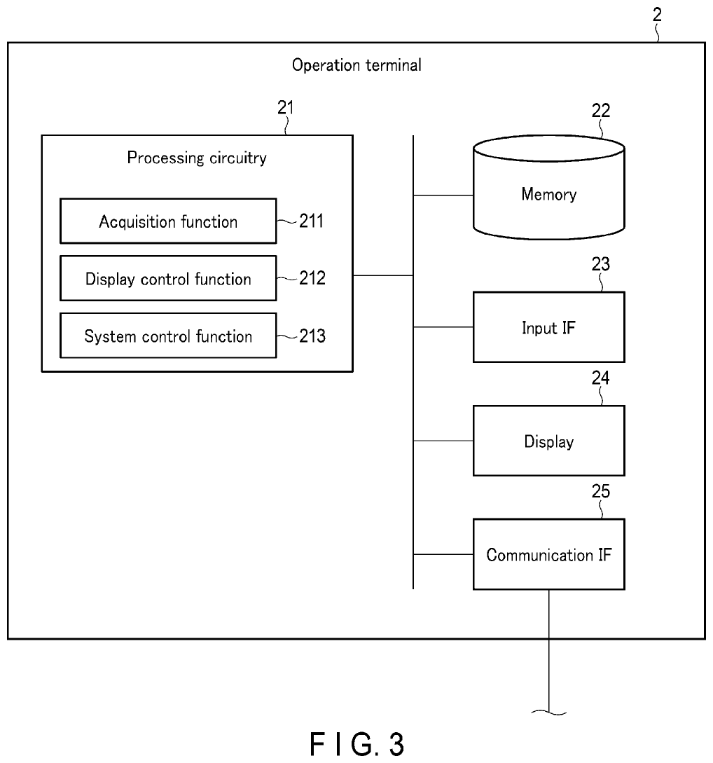

FIG. 3 is a block diagram showing an example of a configuration of the operation terminal 2 according to an embodiment. The operation terminal 2 includes processing circuitry 21, a memory 22, an input IF 23, a display 24, and a communication IF 25 as its components. These components are connected to one another via a bus, which is a common signal transmission path, in such a manner as to be able to communicate with one another.

The processing circuitry 21 is circuitry that controls the entire operation of the operation terminal 2. The processing circuitry 21 of the operation terminal 2 has a hardware configuration similar to that of the processing circuitry 11 of the cell therapy support apparatus 1. The processing circuitry 21 implements each function (an acquisition function 211, a display control function 212, and a system control function 213).

The acquisition function 211 is a function of acquiring various kinds of data or information. For example, the acquisition function 211 acquires display data from the cell therapy support apparatus 1. The display data may include the quantity of tissue sampling determined by the cell therapy support apparatus 1 or a text such as a warning message. The acquisition function 211 is an example of an acquisition unit.

The display control function 212 is a function of causing various kinds of data or information to be displayed. For example, the display control function 212 causes a display image based on the display data to be displayed on the display 24. The display control function 212 is an example of a display controller.

The system control function 213 is a function of controlling various operations performed by the processing circuitry 21. For example, the system control function 213 provides an operating system (OS) for the processing circuitry 21 to implement each function (the acquisition function 211 and the display control function 212). The system control function 213 is an example of a system controller.

The memory 22 is a device for storing various kinds of data or information. The memory 22 of the operation terminal 2 has a hardware configuration similar to that of the memory 12 of the cell therapy support apparatus 1. The memory 22 stores each program for causing the processing circuitry 21 to implement each function (the acquisition function 211, the display control function 212, and the system control function 213). The memory 22 is an example of a storage unit.

The input IF 23 is an interface for receiving various operations from an operator. The input IF 23 converts various operations received from an operator into electric signals, and transmits the electric signals to the processing circuitry 21. The input IF 23 may be a mouse, a keyboard, a button, a panel switch, a slider switch, a trackball, an operation panel, or a touch panel. The input IF 23 is an example of an input unit.

The display 24 is a device for displaying various kinds of data or information. The display 24 may be a liquid crystal display, a plasma display, an organic EL display, or an LED display. The display 24 may be a touch-panel display that also serves as the input IF 23. The display 24 is an example of a display unit.

The communication IF 25 is an interface that communicates various kinds of data or information with each of the components included in the cell therapy support system 100. The communication IF 25 communicates various kinds of data or information with the cell therapy support apparatus 1. The communication IF 25 is an example of a communication unit.

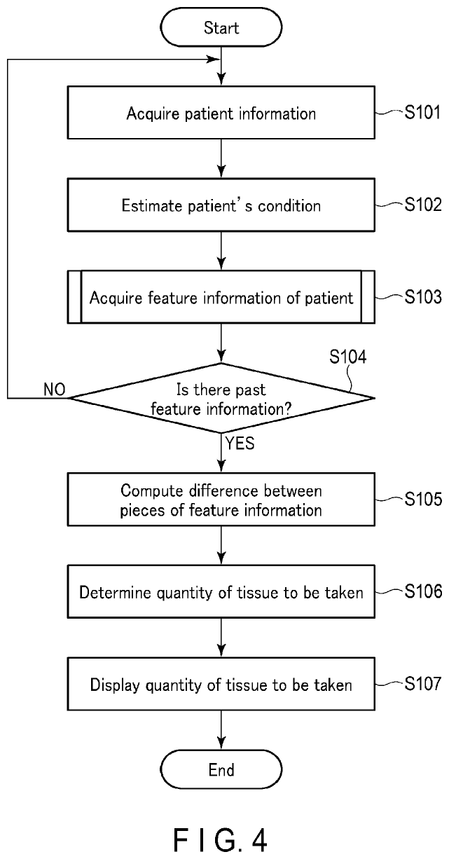

FIG. 4 is a flowchart showing an example of an entire operation of the cell therapy support apparatus 1 according to the embodiment. In this operation example, the cell therapy support apparatus 1 acquires feature information C in a healthy condition and feature information C in an inflammatory condition relating to a target patient. The cell therapy support apparatus 1 determines the quantity of the tissue to be taken from the patient (quantity of tissue sampling) based on a difference between the acquired pieces of feature information C.

(Step S101) First, the cell therapy support apparatus 1 acquires patient information. Specifically, by implementing the acquisition function 111, the cell therapy support apparatus 1 acquires patient information relating to the patient from the medical data storage apparatus 5. The acquired patient information is stored in the memory 12. The acquired patient information includes information indicating the degree of a patient's inflammation (inflammation information).

The inflammation information includes (1) the names of the inflammatory diseases and (2) the test values of the inflammatory diseases. (1) The names of the inflammatory diseases include allergic diseases (e.g., asthma, atopic dermatitis), autoimmune diseases (e.g., autoimmune hemolytic anemia, Graves' disease, Hashimoto's thyroiditis, multiple sclerosis, myasthenia gravis, pemphigus vulgaris, systemic lupus erythematosus, type I diabetes, angiitis), autoinflammatory diseases, etc. (2) The test values of the inflammatory diseases include a C-reactive protein (CRP) value, a sedimentation value, a rheumatoid factor (RF) value, etc.

(Step S102) Next, the cell therapy support apparatus 1 estimates the patient's condition. Specifically, the cell therapy support apparatus 1 estimates the condition relating to the degree of a patient's inflammation based on the patient information acquired in step S101. In particular, the cell therapy support apparatus 1 estimates whether the patient is in a “healthy condition” or in an “inflammatory condition”.

Firstly, if the acquired patient information does not include (1) the names of the inflammatory diseases, the cell therapy support apparatus 1 estimates that there is no degree of a patient's inflammation at all or that the degree of a patient's inflammation is relatively low. That is, the cell therapy support apparatus 1 estimates that the patient is in a “healthy condition”. In contrast, if the acquired patient information does include (1) the names of the inflammatory diseases, the cell therapy support apparatus 1 estimates that the degree of a patient's inflammation is relatively high. That is, the cell therapy support apparatus 1 estimates that the patient is in an “inflammatory condition”.

Secondly, if the acquired patient information includes (2) the test values of the inflammatory diseases and the test values are in a normal range, the cell therapy support apparatus 1 estimates that the patient is in a “healthy condition”. In contrast, if the test values are not in a normal range, the cell therapy support apparatus 1 estimates that the patient is in an “inflammatory condition”.

Regarding the “inflammatory condition”, the cell therapy support apparatus 1 may estimate whether the patient is in a “mildly inflammatory condition” or in a “severely inflammatory condition”. For example, if (2) the test values of the inflammatory diseases are not in a normal range but the test values are relatively close to the normal range, the cell therapy support apparatus 1 estimates that the patient is in a “mildly inflammatory condition”. In contrast, if the test values are relatively far from the normal range, the cell therapy support apparatus 1 estimates that the patient is in a “severely inflammatory condition”. In particular, the cell therapy support apparatus 1 may regard a “mildly inflammatory condition” as a “healthy condition”.

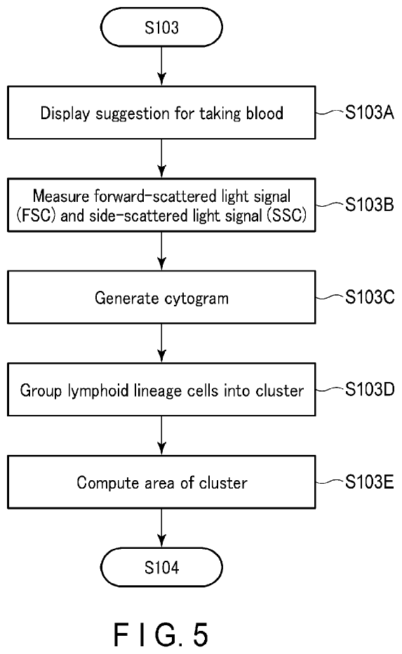

(Step S103) Subsequently, the cell therapy support apparatus 1 acquires feature information C of the patient (see FIG. 5). Specifically, by implementing the acquisition function 111, the cell therapy support apparatus 1 acquires feature information C of the patient in association with the patient's condition estimated in step S102. That is, the cell therapy support apparatus 1 acquires feature information C of the patient in association with the “healthy condition” or the “inflammatory condition” of the patient. Thus, the feature information C of the patient in a “healthy condition” or the feature information C of the patient in an “inflammatory condition” is acquired. The acquired feature information C of the patient is stored in the memory 12.

(Step S104) The cell therapy support apparatus 1 determines whether or not there is past feature information C. Specifically, the cell therapy support apparatus 1 determines whether or not there is past feature information C in addition to the current feature information C acquired in step S103 by searching the memory 12. If there is past feature information C (step S104—YES), the process proceeds to step S105. If there is no past feature information C (step S104—NO), the process returns to step S101. In this case, a series of steps (S101, S102, S103) is performed again at any time point, and new feature information C of the same patient is acquired. The new feature information C acquired is treated as “current feature information C”. The old feature information C already acquired is treated as “past feature information C”.

The current feature information C refers to feature information C of the target patient at the current time. The current feature information C is feature information C of the patient in a “healthy condition” or in an “inflammatory condition”. On the other hand, the past feature information C refers to feature information C of the patient at the past time. The past feature information

C is feature information C of the patient in a “healthy condition” or in an “inflammatory condition”. Preferably, the current feature information C is feature information C of the patient in an “inflammatory condition”, and the past feature information C is feature information C of the patient in a “healthy condition”.

(Step S105) Subsequently, the cell therapy support apparatus 1 computes a difference between the pieces of feature information C. Specifically, the cell therapy support apparatus 1 computes a difference between the current feature information C and the past feature information C of the same patient. In particular, the cell therapy support apparatus 1 computes a difference of the current feature information C from the past feature information C.

(Step S106) Subsequently, the cell therapy support apparatus 1 determines the quantity of tissue sampling. Specifically, by implementing the determination function 112, the cell therapy support apparatus 1 determines a quantity of a tissue to be taken from the patient based on the difference computed in step S105. In particular, the cell therapy support apparatus 1 determines the quantity of tissue sampling by correcting the standard quantity of tissue sampling based on the same difference.

(Step S107) Lastly, the cell therapy support apparatus 1 causes the quantity of tissue sampling to be displayed. Specifically, the cell therapy support apparatus 1 generates display data including the quantity of tissue sampling determined in step S106 and transmits the generated display data to the operation terminals 2. The operation terminals 2 cause a display image based on the transmitted display data to be displayed on the display 24. After step S107, the cell therapy support apparatus 1 ends the series of operations.

FIG. 5 is a flowchart showing an example of a detailed operation of the cell therapy support apparatus 1 according to the embodiment. FIG. 5 shows an example of a detailed operation of step S103 shown in FIG. 4. In this operation example, the cell therapy support apparatus 1 analyzes the patient's blood and thereby computes feature information C indicating the degree of activation of the lymphoid lineage cells contained in the blood. Hereinafter, it is assumed that the cell therapy support apparatus 1 is configured so as to be able to perform flow cytometry.

(Step S103A) First, the cell therapy support apparatus 1 causes a suggestion for blood sampling to be displayed. Specifically, the cell therapy support apparatus 1 generates display data for suggesting blood sampling and transmits the generated display data to the operation terminals 2. The operation terminals 2 cause a display image based on the transmitted display data to be displayed on the display 24.

An operator of the operation terminals 2 takes blood from the patient after observing the display image displayed on the display 24. The operator prepares a blood sample for analysis by, for example, adding a predetermined reagent (e.g., an anticoagulant, a fluorescence-labeled antibody) to the blood taken from the patient. The operator provides the prepared blood sample to the cell therapy support apparatus 1.

(Step S103B) Next, the cell therapy support apparatus 1 measures a forward-scattered light signal (FSC) and a side-scattered light signal (SSC). Specifically, the cell therapy support apparatus 1 analyzes the patient's blood sample via flow cytometry and thereby measures a forward-scattered light signal and a side-scattered light signal for various cells contained in the blood sample. The forward-scattered light signal is an indicator of a cell size. The side-scattered light signal is an indicator of cell complexity.

(Step S103C) Subsequently, the cell therapy support apparatus 1 generates a cytogram. Specifically, the cell therapy support apparatus 1 generates a cytogram with the forward-scattered light signal measured in step S103B as one axis and the side-scattered light signal measured in step S103B as the other axis. The generated cytogram shows the quantities of cells with respect to each of the sizes and each of the complexities of the cells contained in the blood. The generated cytogram is also referred to as a “bivariate histogram”.

(Step S103D) Subsequently, the cell therapy support apparatus 1 groups lymphoid lineage cells into a cluster. Specifically, the cell therapy support apparatus 1 groups the lymphoid lineage cells shown in the cytogram generated in step S103C into a cluster. A model trained by machine learning may be applied to the grouping.

(Step S103E) Lastly, the cell therapy support apparatus 1 computes the area of the cluster. Specifically, the cell therapy support apparatus 1 computes the area of the lymphoid lineage cell cluster formed by the grouping in step S103D. The area of the lymphoid lineage cell cluster is an example of the feature information C. After step S103E, the process proceeds to step S104 (see FIG. 4).

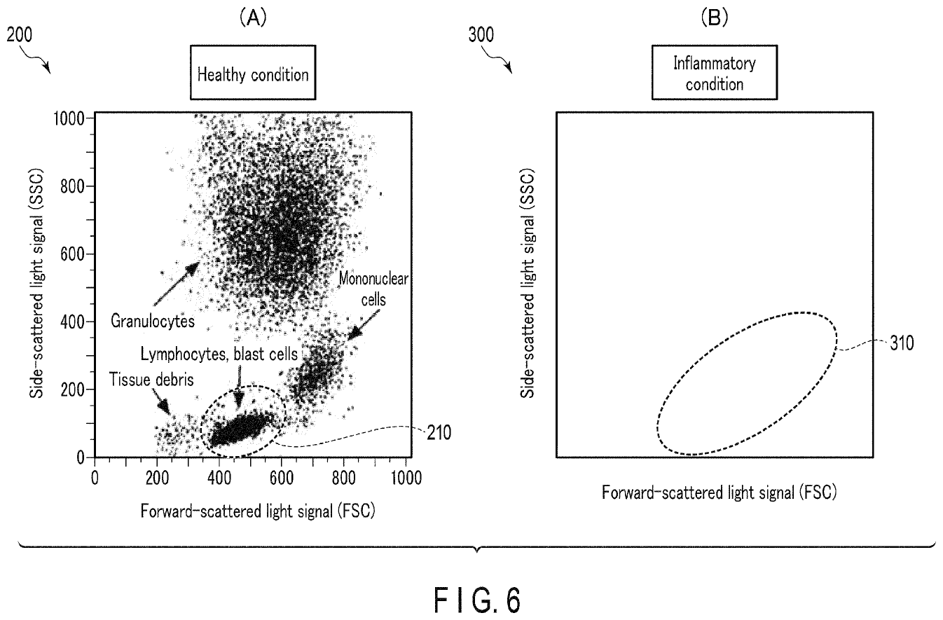

FIG. 6 is a diagram showing examples of cytograms obtained in a healthy condition and in an inflammatory condition according to the embodiment. FIG. 6(A) shows a cytogram 200 of the blood of a patient in a healthy condition. FIG. 6(B) shows a cytogram 300 of the blood of a patient in an inflammatory condition. The horizontal axis of the cytograms 200 and 300 shows a forward-scattered light signal. The vertical axis of the cytograms 200 and 300 shows a side-scattered light signal.

In the cytogram 200, (1) a cluster of granulocytes, (2) a cluster of tissue debris, (3) a cluster 210 of lymphocytes and blast cells, and (4) a cluster of mononuclear cells are observed. Among the four types of clusters, grouping into the cluster 210 is indicated using a broken line. The cluster 210 is a cell population having a forward-scattered light signal value in a range of “about 350 to 600” and a side-scattered light signal value in a range of “about 0 to 200”. The “cluster of lymphocytes and blast cells” is also referred to as a “lymphoid lineage cell cluster”.

In the cytogram 300, a cluster 310 of lymphocytes and blast cells is observed. The cluster 310 in the cytogram 300 is enlarged in the vertical axis direction and the horizontal axis direction, as compared to the cluster 210 in the cytogram 200. In this example, the area of the cluster 310 is more than twice as large as the area of the cluster 210.

As shown in the cytograms 200 and 300, the sizes, complexities, and quantities of lymphoid lineage cells activated in an inflammatory condition are increased, as compared to lymphoid lineage cells not activated in a healthy condition. For example, formation of an oligomer in activated lymphoid lineage cells increases the cell complexity. On the other hand, the cell membranes of activated lymphoid lineage cells have a wavy shape, and the cell membranes of unactivated lymphoid lineage cells have a shape closer to a spherical shape. That is, the feature information C indicating the degree of activation of lymphoid lineage cells may be the sizes, complexities, quantities, or degrees of sphericity of the lymphoid lineage cells.

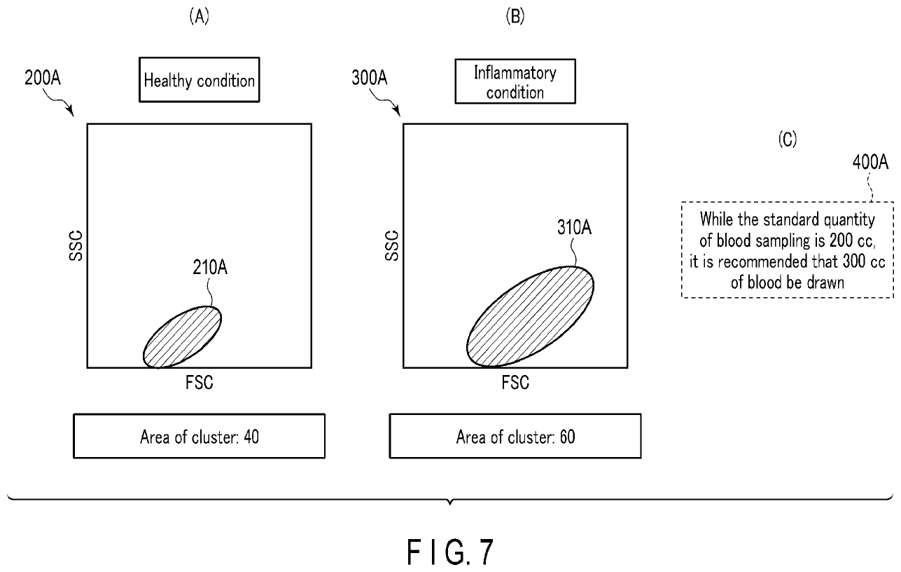

FIG. 7 is a diagram showing a first example of processing performed by the cell therapy support apparatus 1 according to the embodiment. FIG. 7(A) shows a cytogram 200A of the blood of a patient in a healthy condition. FIG. 7(B) shows a cytogram 300A of the blood of a patient in an inflammatory condition. The horizontal axis of the cytograms 200A and 300A shows a forward-scattered light signal. The vertical axis of the cytograms 200A and 300A shows a side-scattered light signal.

In the cytogram 200A, a cluster 210A of lymphocytes and blast cells is observed. The area of the cluster 210A is “40”. In the cytogram 300A, a cluster 310A of lymphocytes and blast cells is observed. The area of the cluster 310A is “60”.

The cell therapy support apparatus 1 computes a ratio (area ratio) between the area of the cluster 210A and the area of the cluster 310A. In this example, the area ratio is computed as “(the area of the cluster 210A): (the area of the cluster 310A)=40:60=2:3”. That is, the area of the cluster 310A is “1.5 times” as large as the area of the cluster 210A.

Herein, it is assumed that a standard quantity of blood to be taken from a patient in a healthy condition is “200 cc”. As mentioned above, the area of the cluster 310A in an inflammatory condition is “1.5 times” as large as the area of the cluster 210A in a healthy condition. Thus, the cell therapy support apparatus 1 corrects the standard quantity of blood sampling by increasing the standard quantity “200 cc” of blood sampling by “1.5 times”. In this case, the corrected quantity of blood sampling is computed as “300 cc”. The cell therapy support apparatus 1 causes the corrected quantity of blood sampling to be displayed on the display 24 of the operation terminals 2.

FIG. 7(C) shows a display image 400A relating to a quantity of blood to be taken from a patient. The display image 400A shows a standard quantity of blood sampling and a corrected quantity of blood sampling. Specifically, the display image 400A shows text stating “While the standard quantity of blood sampling is 200 cc, it is recommended that 300 cc of blood be drawn.”

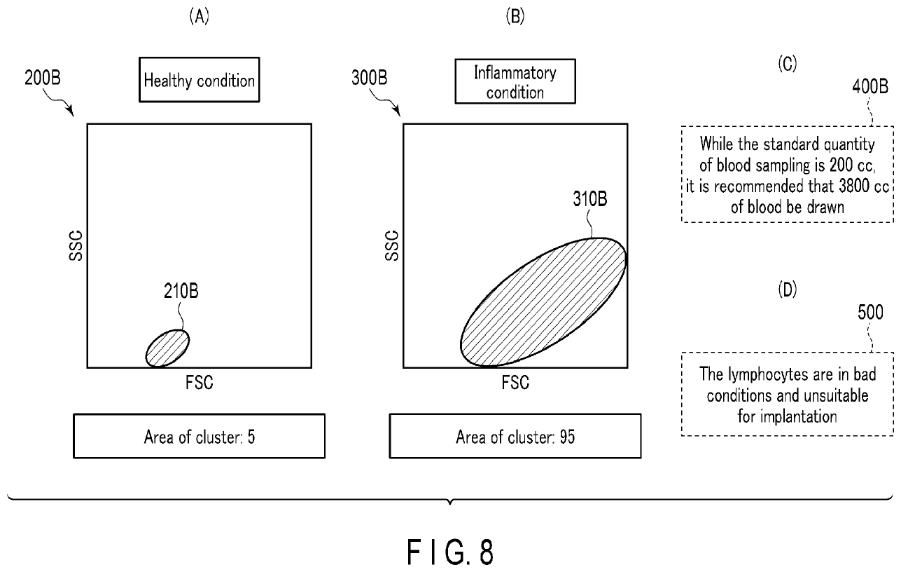

FIG. 8 is a diagram showing a second example of processing performed by the cell therapy support apparatus 1 according to the embodiment. FIG. 8(A) shows a cytogram 200B of the blood of a patient in a healthy condition. FIG. 8(B) shows a cytogram 300B of the blood of a patient in an inflammatory condition. The horizontal axis of the cytograms 200B and 300B shows a forward-scattered light signal. The vertical axis of the cytograms 200B and 300B shows a side-scattered light signal.

In the cytogram 200B, a cluster 210B of lymphocytes and blast cells is observed. The area of the cluster 210B is “5”. In the cytogram 300B, a cluster 310B of lymphocytes and blast cells is observed. The area of the cluster 310B is “95”.

The cell therapy support apparatus 1 computes a ratio (area ratio) between the area of the cluster 210B and the area of the cluster 310B. In this example, the area ratio is computed as “(the area of the cluster 210B): (the area of the cluster 310B)=5:95=1:19”. That is, the area of the cluster 310B is “19 times” as large as the area of the cluster 210B.

Herein, it is assumed that a standard quantity of blood to be taken from a patient in a healthy condition is “200 cc”. As mentioned above, the area of the cluster 310B in an inflammatory condition is “19 times” as large as the area of the cluster 210B in a healthy condition. Thus, the cell therapy support apparatus 1 corrects the standard quantity of blood sampling by increasing the standard quantity “200 cc” of blood sampling by “19 times”. In this case, the corrected quantity of blood sampling is computed as “3800 cc”. The cell therapy support apparatus 1 causes the corrected quantity of blood sampling to be displayed on the display 24 of the operation terminals 2.

FIG. 8(C) shows a display image 400B relating to a quantity of blood to be taken from a patient. The display image 400B shows a standard quantity of blood sampling and a corrected quantity of blood sampling. Specifically, the display image 400B shows a text stating “While the standard quantity of blood sampling is 200 cc, it is recommended that 3800 cc of blood be drawn.”

The cell therapy support apparatus 1 may 1 may determine whether or not the corrected quantity of blood sampling exceeds an upper limit of the blood to be taken from the patient by comparing the corrected quantity of blood sampling with the upper limit. For example, it is assumed that the corrected quantity of blood sampling is “3800 cc” and that the upper limit quantity is “400 cc”. In this case, the cell therapy support apparatus 1 determines that the corrected quantity of blood sampling exceeds the upper limit quantity. The cell therapy support apparatus 1 causes a warning indicating that blood should not be taken from the patient to be displayed on the display 24 of the operation terminals 2.

FIG. 8(D) shows a warning image 500 against blood sampling from a patient. Specifically, the warning image 500 shows a text stating “The lymphocytes are in a bad condition and unsuitable for implantation.” The warning image 500 may be displayed together with the display image 400B or displayed in place of the display image 400B.

Above are descriptions of the cell therapy support apparatus 1 and the cell therapy support system 100 according to the embodiment. According to the embodiment, the cell therapy support apparatus 1 estimates the quality of the stem cells of a patient in a current condition using feature information C (i.e., the degree of activation of lymphoid lineage cells) as an indicator. For example, the cell therapy support apparatus 1 determines the quantity of the tissue to be taken from the patient based on a difference between the feature information C of the patient in a healthy condition and the feature information C of the patient in an inflammatory condition. The cell therapy support apparatus 1 causes the determined quantity of tissue sampling to be displayed on a display.

Thus, the cell therapy support apparatus 1 can support an operator in taking a suitable quantity of a tissue from the patient. Further, the cell therapy support apparatus 1 causes a warning to be displayed on a display if the determined quantity of tissue sampling exceeds an upper limit. Thus, the cell therapy support apparatus 1 can make the operator recognize that a tissue should not be taken from the patient. The operator can feed back, to a treatment program such as cell therapy or apheresis, the quantity of tissue sampling caused to be displayed by the cell therapy support apparatus 1 and whether or not a tissue can be sampled.

As described above, if a patient is in an inflammatory condition, the cell therapy support apparatus 1 increases the quantity of blood to be taken from the patient, in contrast to the case where the patient is in a healthy condition. This is because it is considered that the quality of stem cells degrades when lymphoid lineage cells are activated by an inflammatory disease and damage the stem cells. Thus, the cell therapy support apparatus 1 computes the degree of activation of lymphoid lineage cells of a patient and determines the quantity of blood to be taken from the patient based on the computation result. The cell therapy support apparatus 1 increases the quantity of blood to be taken from a patient if the degree of activation of lymphoid lineage cells of the patient is relatively high (i.e., in an inflammatory condition). As a result, the cell therapy support apparatus 1 can improve the quality of stem cells by taking as many “high-quality” stem cells as possible from a patient.

The embodiment assumes “blood” as an example of a tissue to be taken from a patient. The stem cells contained in the blood may be hematopoietic stem cells, mesenchymal stem cells, or Muse (multilineage-differentiating stress-enduring) cells. The stem cells contained in the blood taken from a patient are implanted in the patient after the cells are processed for implantation.

The embodiment assumes the case where the cell therapy support apparatus 1 may not compute the area of a lymphoid lineage cell cluster of a patient in a “healthy condition”. In this case, the cell therapy support apparatus 1 may instead, compute the area of a lymphoid lineage cell cluster of a patient in a “mildly inflammatory condition”. Alternatively, the cell therapy support apparatus 1 may instead, compute the area of a lymphoid lineage cell cluster of an ordinary human in a “healthy condition”. Thus, the cell therapy support apparatus 1 can compute a difference between the area of a lymphoid lineage cell cluster of a patient in a “healthy condition” and the area of a lymphoid lineage cell cluster of a patient in an “inflammatory condition”.

According to at least one embodiment described above, cell therapy can be supported.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A cell therapy support apparatus comprising processing circuitry configured to:
acquire, for each of a first condition and a second condition differing from each other in a degree of inflammation of a target subject, feature information indicating a degree of activation of lymphoid lineage cells in a tissue containing stem cells of the target subject; and
determine a quantity of the tissue to be taken from the target subject based on a difference between the feature information in the first condition and the feature information in the second condition.

2. The cell therapy support apparatus according to claim 1, wherein the feature information includes at least one of a size, a complexity, a quantity, or a degree of sphericity of the lymphoid lineage cells.

3. The cell therapy support apparatus according to claim 1, wherein the feature information is an area of a cluster including the lymphoid lineage cells in a histogram indicating quantities of cells contained in the tissue with respect to each size and each complexity of the cells.

4. The cell therapy support apparatus according to claim 1, wherein the processing circuitry further acquires a standard quantity of the tissue to be taken from the target subject, and determines the quantity of the tissue to be taken from the target subject by correcting the standard quantity based on the difference.

5. The cell therapy support apparatus according to claim 1, wherein the processing circuitry further acquires an upper limit quantity of the tissue to be taken from the target subject, and causes a warning to be displayed on a display if the quantity of the tissue to be taken from the target subject exceeds the upper limit quantity.

6. The cell therapy support apparatus according to claim 1, wherein
the tissue is blood,
the stem cells include at least one of hematopoietic stem cells, mesenchymal stem cells, or Muse cells, and
the lymphoid lineage cells include at least one of T cells, B cells, or NK cells.

7. A cell therapy support system comprising a cell therapy support apparatus and an operation terminal, wherein
the cell therapy support apparatus comprises processing circuitry configured to:
acquire, for each of a first condition and a second condition differing from each other in a degree of inflammation of a target subject, feature information indicating a degree of activation of lymphoid lineage cells in a tissue containing stem cells of the target subject; and
determine a quantity of the tissue to be taken from the target subject based on a difference between the feature information in the first condition and the feature information in the second condition, and
the operation terminal causes the quantity of the tissue to be taken from the target subject determined by the cell therapy support apparatus to be displayed.

8. A cell therapy support method comprising:
acquiring, for each of a first condition and a second condition differing from each other in a degree of inflammation of a target subject, feature information indicating a degree of activation of lymphoid lineage cells in a tissue containing stem cells of the target subject; and
determining a quantity of the tissue to be taken from the target subject based on a difference between the feature information in the first condition and the feature information in the second condition.
